(19) United States
(12) Patent Application Publication
(10) Pub. No.: US 2011/0033927 A1
(43) Pub. Date: Feb. 10, 2011

(54) METHODS OF GENERATING SMALL-DIAMETER TISSUE ENGINEERED BLOOD VESSELS

(75) Inventors: Marsha Rolle, Worcester, MA (US); Darshan P. Parekh, Framingham, MA (US); Kshama J. Doshi, Framingham, MA (US); Tracy Gwyther, Acton, MA (US)

Correspondence Address: BURNS & LEVINSON, LLP, 125 SUMMER STREET, BOSTON, MA, 02110, US

(73) Assignee: WORCESTER POLYTECHNIC INSTITUTE, Worcester, MA (US)

(21) Appl. No.: 12/752,708

(22) Filed: Apr. 1, 2010

Related U.S. Application Data
(60) Provisional application No. 61/165,726, filed on Apr. 1, 2009.

(51) Int. Cl. C12N 5/071; C12N 5/0775; C12N 5/00

(52) U.S. Cl. 435/353; 435/366; 435/398; 435/402

(57) ABSTRACT

Methods for generating small diameter tissue engineered blood vessels through direct cell seeding onto tubular templates or mandrels, such as fibrin microthreads or collagen-coated silicon tubes, are described.

### CROSS-REFERENCE TO RELATED APPLICATION

This application claims priority to and benefit of co-pending U.S. Provisional Patent Application Ser. No. 61/165,726 filed Apr. 1, 2009 entitled TISSUE ENGINEERED BLOOD VESSELS, which is incorporated by reference herein in its entirety for all purposes.

### BACKGROUND OF THE INVENTION

Cardiovascular disease is among the most significant causes of morbidity and mortality in the United States and other industrialized nations. According to American Heart Association statistics, 13 million Americans have been diagnosed with some form of coronary heart disease and over seven million of these individuals have had a myocardial infarction. In 2002, over 500,000 coronary artery bypass grafting (CABG) procedures and 650,000 angioplasty procedures were performed in the United States to restore blood flow to myocardial tissue supplied by occluded or partially occluded coronary arteries.

The standard of surgical care in CABG is to use the patients' own vessels, most commonly the internal mammary artery or saphenous vein, as the conduit for the bypass procedure. However, in a subset of patients, such as those undergoing reoperation, sufficient autologous arterial or venous graft material may not be available. Synthetic grafts have been used widely for peripheral vascular surgery to replace large vessels, but they are unsuitable for applications such as CABG in which smaller vessels are needed (approximate inner diameter <5 mm), as synthetic grafts of this size rapidly occlude due to thrombosis.

Although various approaches to developing small diameter artificial vascular grafts have been investigated, the ideal blood vessel substitute has not been realized. A variety of techniques have been explored, including cell seeding onto biodegradable polymer scaffolds, formulating tubes from polymerized cell-loaded gels, and creating multi-layered structures by rolling cell sheets onto cylindrical mandrels. Although many of these approaches have resulted in tubular constructs that resemble normal vascular morphology, long-term mechanical stability in vivo has not been consistently achieved. For tissues such as blood vessels which bear a mechanical load, it is challenging to design a material with appropriate strength that also degrades at a rate that matches the rate of cellular growth and tissue remodeling. If the cells are unable to create a matrix to replace the original scaffold material and provide sufficient structural integrity to the tissue as it degrades, the results can be catastrophic.

Given the aforementioned limitations associated with using exogenous scaffolds to engineer vascular grafts, alternative approaches in which tissue engineered blood vessels (TEBV) are generated using only cells, and the extracellular matrix (ECM) they produce, have been explored. For example, previous studies have shown that cells that adhered to and encapsulated a silicone mandrel implanted in the peritoneal cavity formed a fibrous neotissue within two weeks that could function as a vascular conduit when implanted into the arterial circulation of the host animal. Although the autologous source of cells and the short incubation period required to generate these vessels is impressive, implantation of the graft nucleating material requires an invasive surgical procedure, which may limit its utility in clinical practice. Alternatively, cell-derived TEBV generated from sheets of cells cultured under conditions that promote ECM synthesis rolled around a tubular support have been implanted in a small group of dialysis patients as arterio-venous shunts. The results of these studies demonstrate that, as in normal vascular development, the cell itself is capable of generating a three-dimensional tissue strong enough to retain its shape even in the presence of the stresses associated with pulsatile arterial blood flow. However, to achieve this strength, cell sheets are cultured for six weeks, followed by wrapping the sheets around a mandrel to form a tube, which then requires an additional ten weeks of culture to allow fusion of the cell layers and formation of a cohesive vascular tissue.

### BRIEF SUMMARY OF THE INVENTION

This invention is directed to methods of generating small diameter tissue engineered blood vessels through direct cell seeding onto tubular templates or mandrels, such as fibrin microthreads (using a non-adhesive v-shaped chamber), or collagen-coated silicon tubes (using a “hanging drop” cell suspension). The methods described herein take place in a shorter time period than has been previously recorded, and result in microvessels that are thick enough to bear mechanical load, leading to long-term stability.

### BRIEF DESCRIPTION OF THE DRAWINGS

FIG. 1 is a schematic of cells seeded onto a tubular mandrel according to an embodiment of the present invention;

FIG. 2 is a schematic of the preparation and dimensions of the v-shaped chambers of this invention;

FIG. 3 is a schematic of the hMSC seeding onto fibrin microthreads in v-shaped chambers of this invention;

FIGS. 4a-d are photographic illustrations of stained fibrin microthreads;

FIG. 5 is a graph of hMSC cell attachment in v-shaped agarose chambers;

FIGS. 6a-f are photographic illustrations of cell attachment on UV cross-linked fibrin microthreads;

FIGS. 7A-C are photographic illustrations of stained images of UV cross-linked fibrin microthreads at three, five and seven days;

FIG. 8 is a graph of cell layer thickness as a function of culture time on cross-linked fibrin microthreads;

FIG. 9 is a graph of cell attachment as a function of culture time on cross-linked fibrin microthreads;

FIGS. 10a-c are a schematic of a cell-seeding procedure according to another embodiment of the present invention;

FIGS. 11a-b are photographic illustrations of a collagen gel coating mold;

FIG. 12 is a photographic illustration of a collagen gel coating procedure;

FIGS. 13a-b are photographic illustrations of the casting of silicon support rings;

FIGS. 14a-7b are photographic illustrations of the molding silicone support rings;

FIGS. 15a-b are photographic illustrations of the fabrication of cell-seeding assemblies;

FIGS. 16a-b are photographic illustrations of the final cell-seeding assembly;

FIG. 17 is another schematic of a cell-seeding procedure of FIGS. 6a-c;

FIG. 18 is a graph of cell growth as a function of culture time on collagen-coated silicone mandrels; and

FIGS. 19a-e are photographic illustrations measuring cell tube thickness.

### DETAILED DESCRIPTION OF THE INVENTION

### Example 1: Cell Seeding on Tubular Mandrels

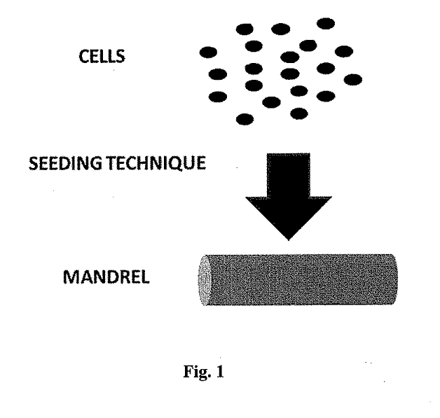

In one embodiment of the present invention, cells were directly seeded onto tubular mandrels (FIG. 1) using a non-adhesive v-shaped chamber. The tubular mandrels can be made of any suitable material for cells to adhere to which will degrade over time, leaving a lumen. In this example, fibrin microthreads were used as tubular mandrels which were anchored in v-shaped chambers cast from agarose. Cells do not adhere to agarose, therefore the seeded cells are compelled to attach only to the microthreads.

To create the v-shaped agarose chambers, two grams of agarose (SeaKem LE Agarose, Lonza) were added to 100 ml of Dulbecco's Modified Eagle's Medium (DMEM, Cellgro) to generate a 2% solution, which was then autoclaved for sterilization. The 2% agarose solution was then poured onto a pyramid-shaped template and allowed to gel for 45 minutes. The template was then removed, and the resulting v-shaped agarose chamber was transferred to cell culture wells using spatula. DMEM was filled in the culture wells around the v-shaped chambers prior to cell seeding. The dimensions of the chamber were approximately 1.5 cm×0.75 cm×0.5 cm. The sterilized microthreads were anchored at the bottom of the v-shaped chamber using triangular slabs made from autoclaved polydimethyl siloxane (PDMS) as weights.

The fibrin microthreads were created using the protocol described in Cornwell et al. (Cornwell, K, Pins G: Discrete crosslinked fibrin microthread scaffolds for tissue regeneration. J. Biomed Mater Res A 2007; 82:104-112, incorporated by reference herein). Briefly, 70 mg/ml of fibrinogen solution (Bovine plasma, Sigma, St Louis, MO F4753) was prepared in HEPES buffered saline (HBS, 20 mM HEPES, 0.9% NaCl). Thrombin was reconstituted at 6 U/ml in 40 mM CaCl₂ solution. These final stocks of fibrinogen and thrombin were then co-extruded through polyethylene tubing with an inner diameter of 0.38 mm into a bath of 10 mM HEPES solution. The fibrin microthreads were then removed from the bath suspended at their two ends and allowed to dry under the tension of their own weight. After drying, the microthreads were stored in a dessicator at room temperature until use. The average diameter of the microthreads in their hydrated state was 65 μm.

Bone marrow-derived human mesenchymal stem cells (hMSC) (Lonza) were seeded in a 150 mm Petri dish at 5,000 cells/cm² in 20 ml of media. The cells were used between passages 7 to 11 for studies. The hMSC were cultured in Mesenchymal Stem Cell Growth Medium (MSCGM) consisting of Mesenchymal Cell Growth Supplement (MCGS), L-Glutamine and GA-1000 (MSCGM™ BulletKit, Cat# PT-3001, Lonza). At 90% confluency, the cells were trypsinized using 0.25% trypsin EDTA solution (CellGro, Mediatech Inc.) and a cell count was performed using trypan blue exclusion method.

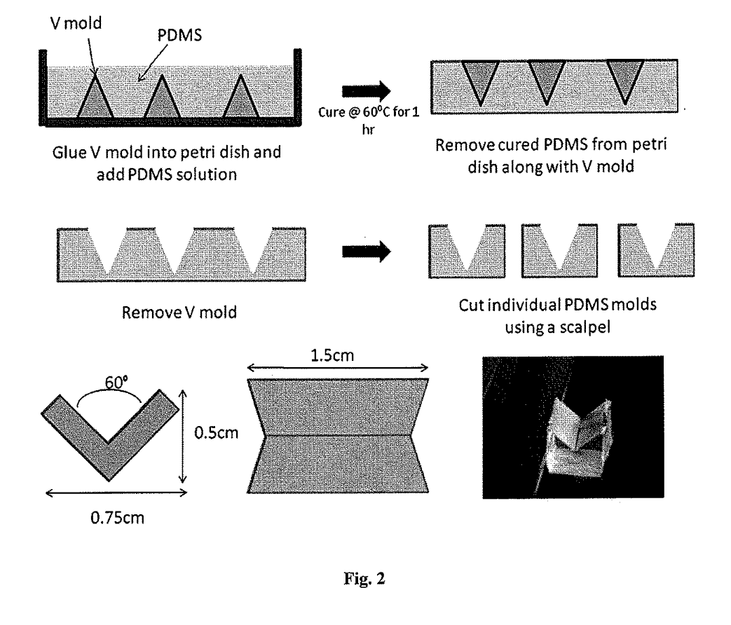

To create the v-shaped agarose chambers cast from PDMS, polycarbonate molds machined in the shape of a pyramid served as a negative template. PDMS was prepared as a mixture of silicone elastomer base and silicone elastomer curing agent in the ratio of 10:1. The PDMS was then degassed. The polycarbonate molds were glued on the inside of the Petri dish, with the apex of the molds facing up. The dish was then covered with the degassed PDMS such that the apexes of the molds were immersed into the PDMS. The setup was then heated in an oven at 60° C. for 60 minutes. The cured PDMS, along with the polycarbonate molds, was then removed from the Petri dish. The molds were removed manually and PDMS v-shaped channels remained (FIG. 2). The final dimensions of the seeding chamber were 1.5 cm×0.5 cm×0.75 cm.

The PDMS v-shaped channels were autoclave sterilized. The fibrin microthreads were then glued along bottom of the channel lengthwise using silicone adhesive (Silicone medical adhesive type A, Dow Corning). It was important to make sure that the microthread was resting at the bottom of the channel before securing in position using glue at the two ends. The glue was allowed to dry onto the microthreads in the channel for 48-72 hours. The microthread was then subjected to a series of rinsing steps for sterilization: 15 min PBS, 45 min 70% ethanol, 3×15 min PBS. After sterilization, the microthreads were directly used for seeding without allowing them to dehydrate.

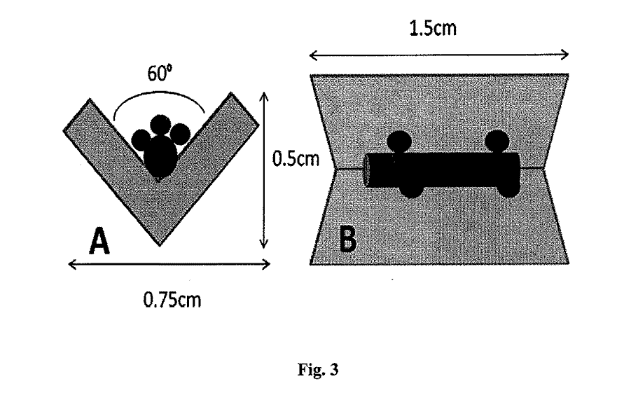

The hMSC then were trypsinized and resuspended at the required concentration of 50,000 cells in 100 μl medium as described above. The PBS from the v-shaped channels was aspirated and 100 μl of the hMSC solution was added to the channels, making sure the seeding volume is evenly distributed along the microthread length. The hMSCs were allowed to seed onto the microthreads for four hours at 37° C. as shown in FIG. 3. After four hours of seeding, the microthreads were analyzed for cell quantification, proliferation, and viability.

For long term culture, the hMSC solution was aspirated from the v-shaped channels post seeding. The sample was then rinsed in PBS to make sure that only the attached cells (during seeding) are cultured whereas the clinging and loosely attached cells are washed off. Each sample was then secured into a well of a 6-well plate using sterile silicone vacuum grease (976V High vacuum grease, Dow Corning). Fresh media (7 ml) was then added into each well, making sure the sample was completely covered in media. The samples were then cultured for three, five and seven days. Media was changed every third day.

Fibrin microthreads were crosslinked as per the protocol described by Cornwell et al. using a CL-1000 ultraviolet crosslinker (UVP, Upland, Calif.). The crosslinking device consisted of a bank of 5-8 W UV tubes that produced radiation to crosslink the microthreads. Strands of microthreads were placed on a reflective surface of aluminum foil which was centered at a distance of 11 cm from the UV tubes. The exposure time was 20 minutes and hence resulted in a total energy of 8.5 J/cm².

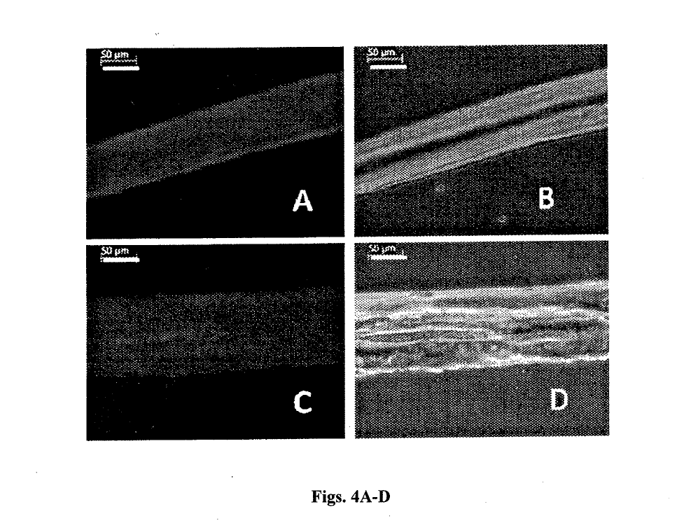

To visualize hMSC attachment to fibrin microthreads, the hMSC-seeded microthreads were removed from the seeding chambers at their respective time points using pointed forceps and scissors. They were thoroughly rinsed in PBS to get rid of any clinging or loosely attached cells. The microthread sample was then transferred onto a glass slide and stained with the Hoechst dye for five minutes, and protected from light during staining. After staining for five minutes, the Hoechst dye was removed and the microthread was washed with PBS three times. The microthread was mounted using a cover slip and mounting medium (Cytoseal, Cat#81678, Richard-Allan Scientific). Images were obtained using a Leica DM IL inverted fluorescent microscope in order to analyze the number of nuclei and morphology of attached cells (FIGS. 4a-d).

To quantify the number of cells adhered to the fibrin microthreads, The CyQuant NF assay (Invitrogen, Cat#C35006/C35007) was used due to its reported high sensitivity for quantifying cell numbers in the linear range of 20-20,000 cells. It is simple in that it does not require any washes, cell lysis or prolonged incubation. The working principle of the assay is that the CyQuant NF dye permeates through the cell membrane and binds to the DNA. On binding to the DNA it emits fluorescence which can be measured. As DNA content represents cell number, this assay is effective for cell quantification from a sample and can be completed in as little as an hour. The cell quantification assay showed that 1654±22 cells attached per 1 cm fibrin microthread sample (n=3). Thus, hMSCs adhere rapidly to fibrin microthreads, which can be used for cellular microvessel generation. After only four hours of seeding with only 10,000 cells, hMSCs adhered uniformly across the length of the microthread in the v-shaped agarose chamber. Based on the number of seeded cells and the number of attached cells based on cell quantification, the seeding efficiency is ˜16%.

A total of three samples of fibrin microthreads were seeded with hMSCs using the v-shaped chambers and phalloidin and Hoechst staining were performed. Images from different regions along the length of each microthread were acquired in order to assess the cell attachment and distribution. Confocal microscopy was used to obtain images of different regions of a microthread sample to visualize the two stains in the same image.

The Hoechst images of four different sections of the microthread indicate that the cells are more evenly distributed throughout the microthread length as compared to the hanging drop method. The count from Hoechst-stained images was an average of 5680±850 cells per cm of microthread. Also, there was no evidence of formation of clusters that was observed using the hanging drop seeding method. Phalloidin staining indicates complete coverage of the microthread by cells after only four hours of seeding.

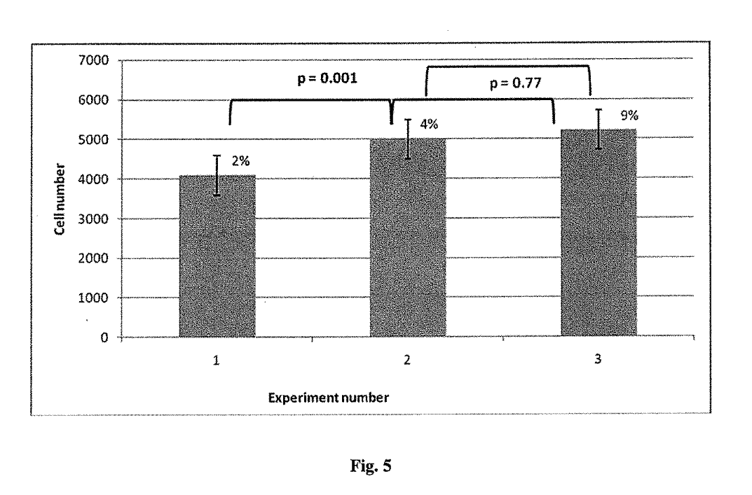

Three independent cell seeding experiments were performed using the v-shaped chambers. Three samples from each of these experiments were subjected to the CyQuant NF assay to quantify the number of cells attached. FIG. 5 shows a cell attachment of 4,007±100 cells, 5230±205 cells and 5,000±223 cells per cm of microthread in the three experiments involving seeding in the v-shaped chambers. The coefficient of variance observed in these experiments was 2%, 4% and 9%, respectively.

Three fibrin microthreads were seeded with hMSCs in the v-shaped chambers for four hours and then cultured for three days and cell number on the microthreads was quantified by CyQuant NF assay. The cell count from CyQuant after three days of culture was 8,880±432 cells per microthread, resulting in a coefficient of variance of 5% of the mean cell count.

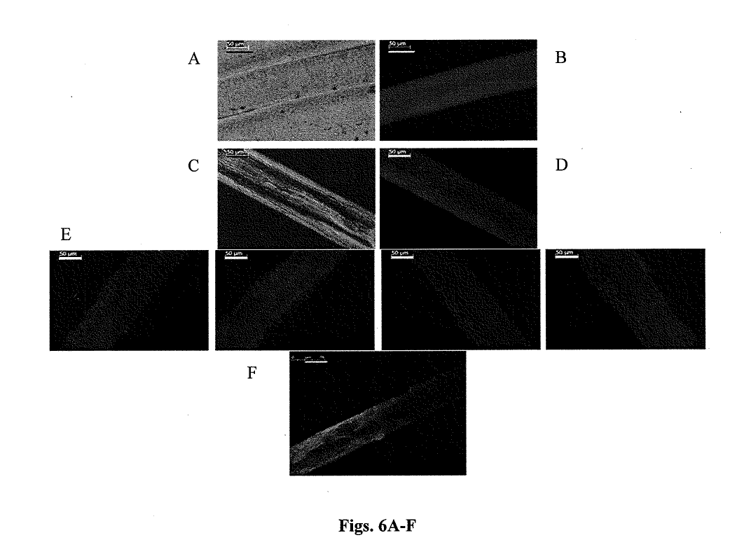

To prevent microthread degradation, microthreads were crosslinked by UV irradiation to increase their mechanical strength. The same cell seeding procedure was performed as described for hMSC seeding and culture of uncrosslinked microthreads. The images in FIGS. 6 a-f show that the hMSC attachment on UV crosslinked microthreads was similar to that of the uncrosslinked microthreads. The four Hoechst-stained sections of the sample in FIG. 6e show even cell attachment at different regions of the microthread. There was no evidence of formation of cell clusters after seeding. This was further confirmed by taking a z-stack using the confocal microscope. For taking a z-stack, the microthread sample in consideration was scanned from one side to the other and images are taken at every predetermined interval. These images were then stacked to give a composite image of the whole microthread thickness.

Three UV crosslinked fibrin microthreads seeded with hMSCs in the v-shaped chambers for four hours were quantified by CyQuant NF assay. The cell count by CyQuant was 4950±210 cells that show a standard deviation of 4% with the sample group.

After seeding for four hours, the attached hMSCs were allowed to culture on the UV crosslinked fibrin microthreads for three, five and seven days each. Three samples at each time point were co-stained with Hoechst and phalloidin to analyze cell attachment and distribution. Confocal microscopy was used to capture and merge the two stains in the same image. Phase images were also taken for comparison.

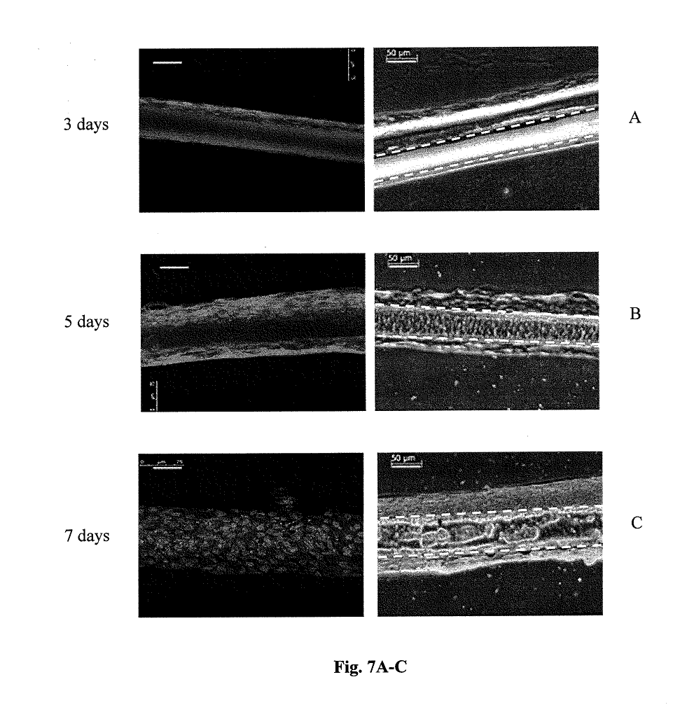

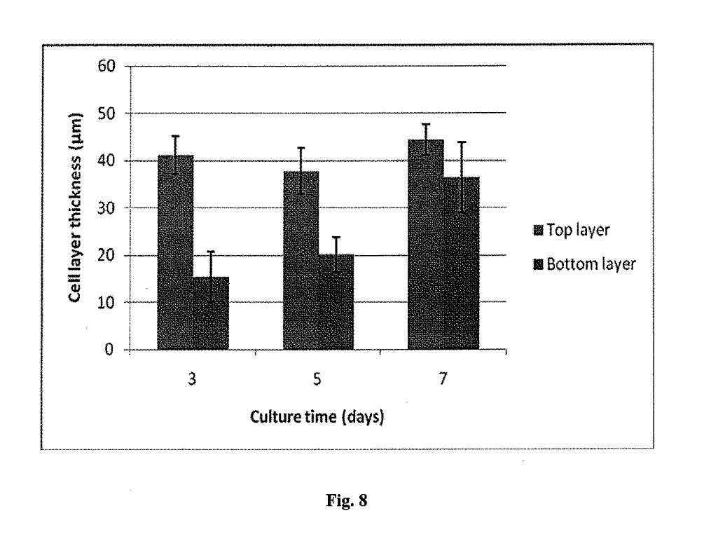

The Hoechst-phalloidin co-stained as well as phase images (FIG. 7) show an increase in cell growth with the culture period. After three days of culture, one side of the microthread appeared to have fewer cells, similar to what was observed after four hours of seeding. However, by day three, the hMSCs seemed to be forming multi-layers in the areas where they attached after four hours of seeding. After five days of culture, the cells started migrating to areas of the microthread which lacked cell attachment after seeding and three days of culture. There was a simultaneous increase in the wall thickness observed by the increment in attached cell layers around the microthread. Eventually at seven days, the microthread was completely confluent with the cells. The two dotted lines in each phase image represent the boundary of the microthread. The attached cell layers above and below the lines suggest uniformity of cell attachment along the circumference of the microthread. However, the difference in the thickness of the top and bottom cell layers in phase images of FIG. 7 indicates that the cells had attached over the top surface of microthread that was exposed to seeding. Nonetheless, there is an increase in the bottom layer thickness over culture period as indicated by FIG. 8. With the culture period, the difference in thickness of top and bottom cell layers decreases and cell growth tends to become more uniform circumferentially.

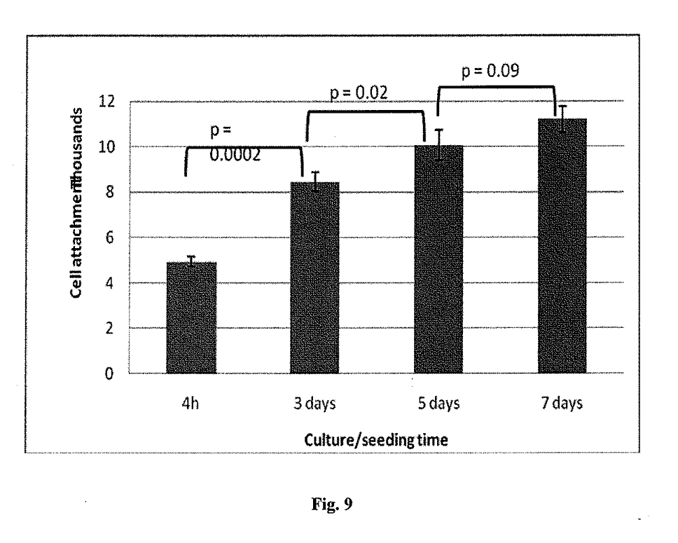

Three samples at each culture period were assayed by CyQuant NF in order to quantify the number of cells per microthread at each time point. The average cell count and deviation were plotted for each time point and observed over the culture period, shown in FIG. 9. Observations showed 8,460±420 hMSCs after three days in culture, 10,000±670 hMSCs after five days and 11,200±581 hMSCs after seven days. The coefficient of variance observed were 5%, 6.65% and 5.2% respectively for each of these time points. One way ANOVA between four hour seeding and three day culture gave a p-value of 0.0002 indicating the cell count at three days is significantly greater than at four hours. Similarly, after five days, there was a significantly greater cell number than after three days (p=0.02) but the cell counts at seven days were not significantly greater than five days (p=0.09). This result may indicate a decrease in the rate of cell growth at the seven-day time point. The results are consistent with the images in the previous section which show an increase in cell number on fibrin microthreads as a function of time in culture.

### Example 2: Cell Seeding on Collagen-Coated Silicon Tubes

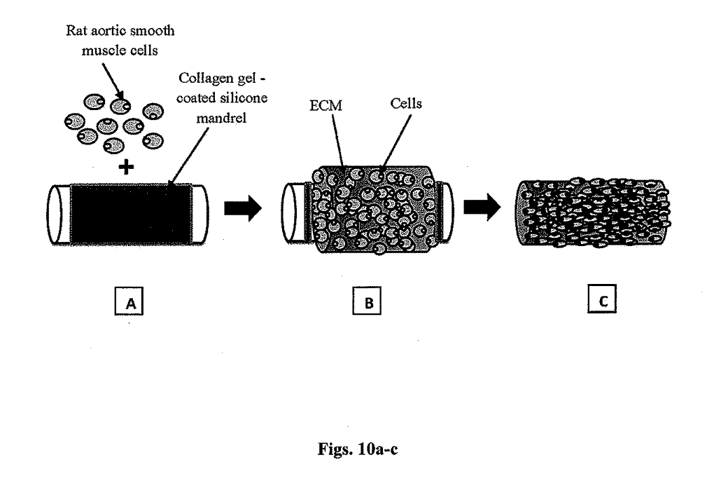

In another embodiment of the present invention, cells were directly seeded and cultured on a collagen-coated tubular mandrel as shown conceptually in FIGS. 10a-c. The collagen gels were made as previously described by Puchelle et al. (Puchelle et al., Proliferation, differentiation and ciliary beating of human respiratory ciliated cells in primary culture, Cell Tissue Res, 1991, 264:49-55, incorporated by reference herein in its entirety). A 1 mg/ml collagen gel solution was prepared by mixing the 5X DMEM solution with 1 N sodium hydroxide (Cat# 5062-8576, Agilent Technologies), 0.1 N acetic acid (glacial acetic acid, Cat# 42322-5000, Acros) and rat tail collagen gel type I (Cat# 354236, BD Biosciences), on ice. A 5X DMEM solution was made by mixing DMEM powder, (Conc. 13.49 g/L, Cat# 50-003-PB, CellGro, Mediatech Inc.) with sodium bicarbonate (Conc. 3.7 g/L, Cat# 5-5761, Sigma.Aldrich) in distilled water. The solution was sterile-filtered using a 0.2 μm syringe filter (Acrodisc 13 mm syringe filter, Cat# 2012-03, Pall Corporation) before injecting into a custom mold.

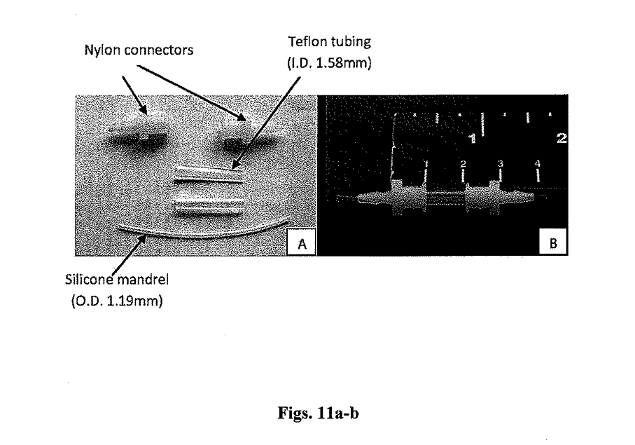

The custom mold was made by using 1.58 mm LD. Teflon tubing (PTFE tubing, Item# 06605-28, Cole Parmer), a 1.19 mm O.D. silicone tube (Silastic Laboratory tubing, Cat# 508-003, Dow Coming) and a pair of nylon connectors (Cat# LCN-F093-C, Small Parts Inc.) as shown in FIG. 11a. These molds were assembled by cutting approximately 3 cm long pieces of the Teflon tubing into two halves through the center using a scalpel blade. The two halves were then coupled together using two nylon connectors. Then, a 5 cm long piece of silicone tubing was inserted into the Teflon tubing through the connectors to form the final coating mold shown in FIG. 11b.

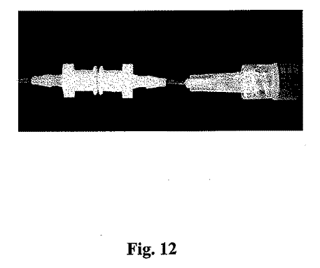

Coating was achieved by injecting the prepared collagen gel solution into the custom molds using a 30 gauge hypodermic syringe needle (PrecisionGlide needles, Cat# 305106, BD) as shown in FIG. 12. The collagen gel solution was allowed to polymerize for one hour at 37° C. in an incubator. After one hour of polymerization, the gelled collagen-coated silicone mandrels were removed from the molds and dialyzed in sterile distilled water overnight to remove salts from the gel. Following dialysis, the coated silicone mandrels were air-dried overnight to form a dehydrated film of collagen around the silicone mandrel barely visible to the naked eye. After dehydrating the collagen gel coating on the silicone mandrel, its presence was confirmed in a subset of samples by staining the coated mandrels with Trypan blue solution (0.4% wlw, Cat# 25-900-CI, CellGro, Mediatech Inc.) for 10 minutes. Trypan blue is a vital stain and it binds to connective tissue elements like collagen, reticulin and elastic fibers.

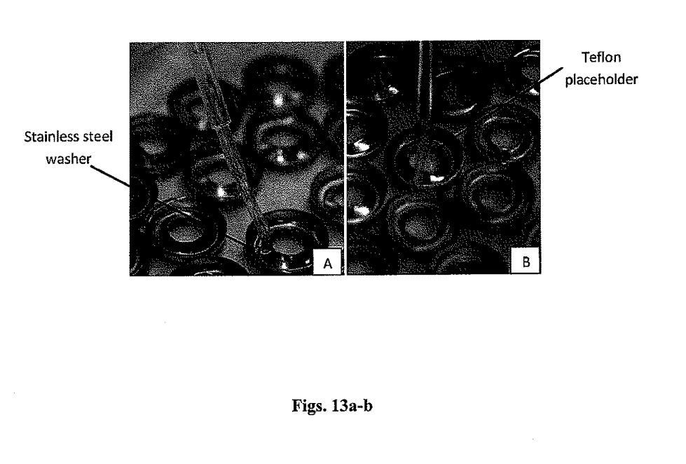

Polydimethylsiloxane (PDMS), a silicon based organic polymer, was used to fabricate a silicone support ring for the cell seeding assembly. PDMS was prepared by mixing 1:10 curing agent to elastomer base (184 Sylgard silicone elastomer base kit, Dow Corning). This solution was then transferred into stainless steel washers (#12 Finishing washers, Item# 3273, Home Depot) using a liquid dropper as seen in FIG. 13a. Then, a 1.6 mm O.D. Teflon tube used as a placeholder (Cat# SLTT-16, Small Parts) was positioned along the diameter of the washer to create grooves for the collagen-coated silicone mandrels to fit (FIG. 13b).

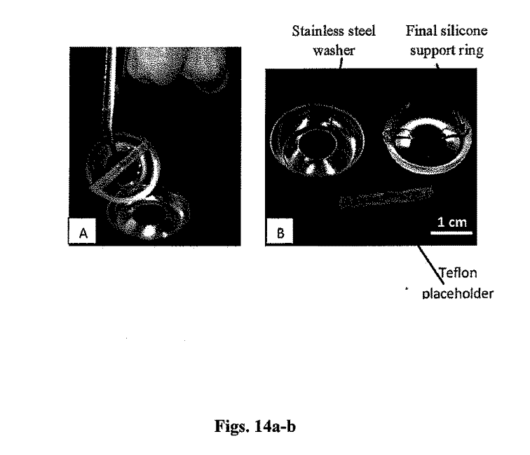

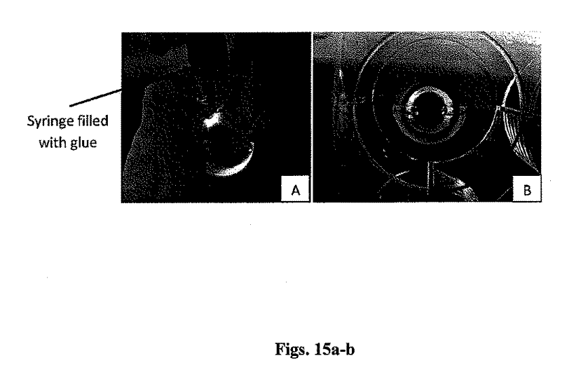

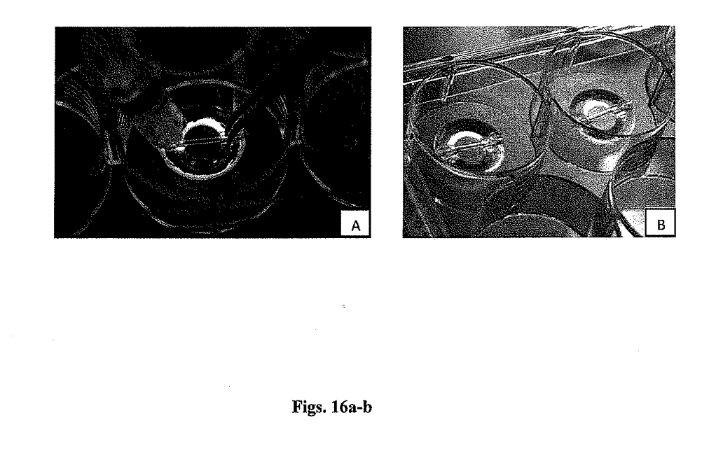

After one hour of polymerization at 60° C., the molded silicone support rings and the Teflon placeholders were removed from the stainless steel washers as seen in FIGS. 14a-b. Individual support rings were then autoclaved for one hour at 121° C. and 19 psi pressure. After autoclaving, each of the support rings were glued into wells of a standard 6-well plate (Multiwell, 6 well tissue culture dish, Cat# 3026, BD) using a 3 ml syringe filled with sterile silicone glue (Silastic Silicone Medical Adhesive Type A, Dow Corning), as seen in FIGS. 15a-b. To prepare for seeding, the collagen-coated silicone mandrels were glued into the grooves created by the Teflon placeholder on the silicone support rings using silicone glue as seen in FIG. 16a. The glue was allowed to air dry for 24 hours. This is important since the silicone adhesive releases acetic acid vapors in the initial 24 hours of application, which is toxic to cells (Product data sheet, Silastic Silicone Medical Adhesive 30 Type A, Dow Corning). Preparation of seeding assemblies was performed in the biosafety cabinet.

Adult rat aortic smooth muscle cells were plated at a density of 6,500 cells/cm² in a 150 mm Petri dish (Cellstar tissue culture dishes, Cat# 639160, Greiner Biosciences) and cultured in growth medium consisting of 1X DMEM (1X DMEM, Cat# 15-017-CV, CellGro, Mediatech Inc.) supplemented with 10% fetal bovine serum (FBS, Cat# A15-201, PAA Laboratories), 1% nonessential amino acids (NEAA, Cat# 25-025-CI, CellGro, Mediatech Inc.), 1% Glutamax (Cat# 25-015-CI, CellGro, Mediatech Inc.) 1% sodium pyruvate (Cat# 25-000-CI, CellGro, Mediatech Inc.) and 1% penicillin/streptomycin (Cat# 30-002-C1, CellGro, Mediatech Inc.) until they reached 80% confluency. The cells were then trypsinized, counted and resuspended at a concentration of 1 million cells/ml in growth medium.

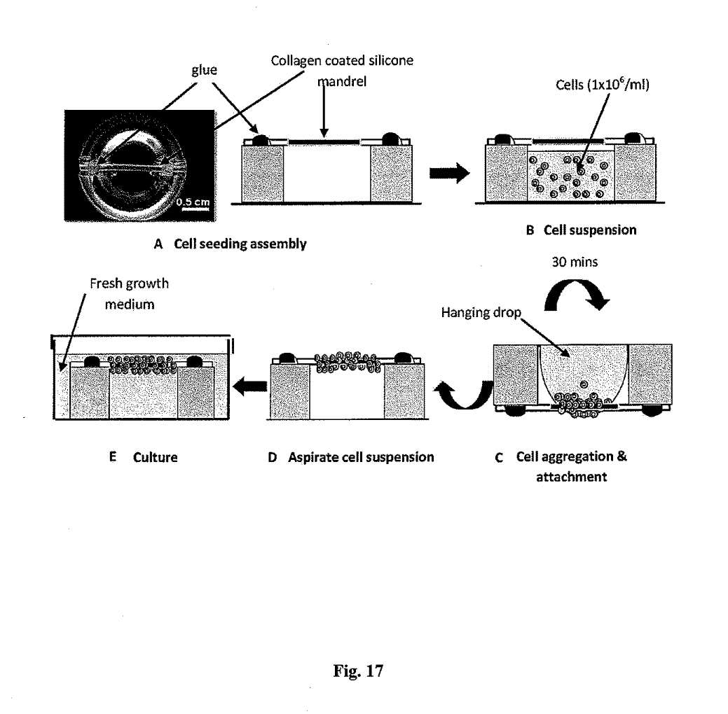

After preparing the cell seeding assemblies (FIG. 16b), and prior to seeding, the collagen-coated silicone mandrels were rehydrated with 400 μl of standard growth medium for 30 minutes. For cell seeding, 280 μl of the prepared cell suspension (280,000 cells total) was pipetted into the center of each silicone support ring as seen in FIG. 17. The culture dishes were then inverted and incubated at 37° C., 5% CO₂ for 30 minutes. The cell suspension forms a droplet and the cells aggregate at the bottom of this droplet which allows them to contact the surface of the coated silicone mandrel. After 30 minutes of incubation in this inverted position, the culture dish was turned upright and the samples were rinsed twice with 1X PBS to remove the unattached cells.

After rinsing with PBS, 6 ml of fresh growth media was added to each well and the samples were allowed to culture at 37° C., 5% CO₂. Following seeding, cell attachment on collagen-coated silicone mandrels was verified by taking phase contrast images using a Leica DM IL inverted microscope. The images of the cell-seeded mandrels were compared with the unseeded silicone mandrels immediately after 30 minutes of seeding. This observation tested the necessity of collagen gel coating in tissue tube formation by direct cell seeding method. A schematic of the entire procedure is shown in FIG. 17.

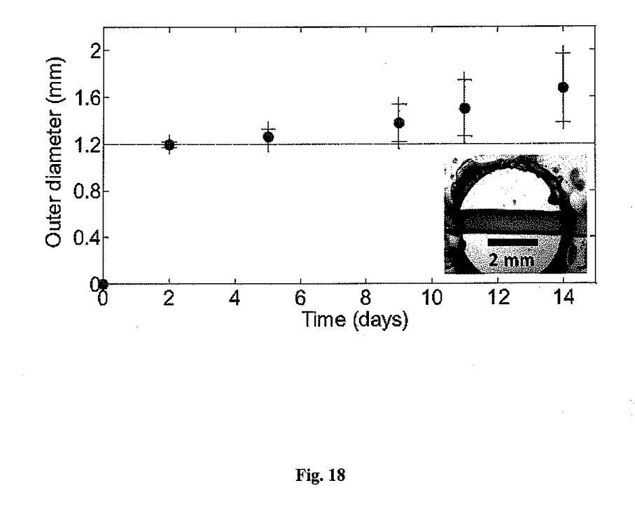

The growth of the cell tubes was monitored using a high resolution CCD camera (DVT 600, Cognex) and image acquisition software (Framework 2.2, Cognex). This system enabled noninvasive quantification of cell tube thickness. At each time point, samples were removed from the culture incubator and a digital image was captured (see inset, FIG. 18). The outer diameter of the cell-tube samples was measured in ten locations along the length of the sample. Outer diameter values are expressed as mean ±S.E.M.

After 14 days in culture, engineered tissue tubes were harvested fresh or after fixing in 10% neutral buffered formalin (30 minutes) from the mandrel support for purposes of biomechanical testing and histological analysis, respectively. The silicone mandrels were first removed from the silicone support ring using a scalpel. Then, both the ends of the silicone mandrel were stretched in opposite directions using a pair of forceps to detach the tissue tube from the mandrel. The tissue tube was then slid off the silicone mandrel for further characterization.

For structural characterization, the vascular tissue tubes were fixed in 10% neutral buffered formalin for 30 minutes after 14 days of culture. The fixed samples were removed from the tubular mandrels as described in Section 3.6, cut into two halves at the centre, processed and embedded vertically in paraffin. After embedding, the tissue tubes were sectioned using a microtome (5 μm) and mounted on glass slides (Cat # 48311-703, V)WR). The slides were then baked at 60° C. for 30 minutes.

The measured outer diameter (FIG. 18) is the sum of the starting thickness of the silicone tube and the thickness of the cell growth around it. By subtracting the O.D. value from the silicone tube diameter and dividing by half, it was determined that the average wall thickness at 14 days was 0.24±0.1 mm.

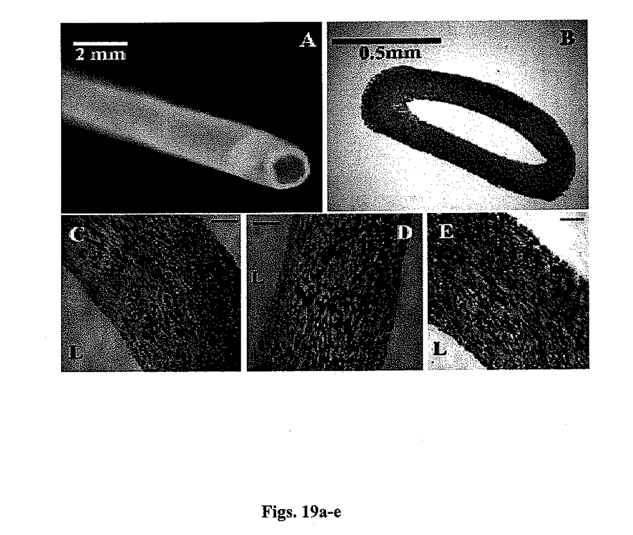

As seen in FIGS. 19a-e, the histology of the constructs revealed viable cellular tissue with circular alignment of the cells along the outer edges of the constructs. Very little collagen was observed within the cell tubes (FIG. 19d), but there was an abundance of glycosaminoglycan staining (blue, FIG. 19e). Wall thickness values measured from histological sections were 0.263±0.59 mm (n=3), comparable to values calculated from non-invasive measurements. These results provided evidence that in a period of 14 days, it is possible to develop a three dimensional tubular structure with contiguous layers of cells that is strong enough to withstand handling.

What is claimed is:

1. A method of generating small-diameter blood vessels, the method comprising:
anchoring a sterilized fibrin microthread in a v-shaped chamber;
seeding a number of cells on the fibrin microthread for a period of time, such that the cells attach uniformly onto the fibrin microthread, resulting in a tissue engineered blood vessel.

2. The method of claim 1, wherein the diameter of the fibrin microthread is approximately 100 μm.

3. The method of claim 1, wherein the length of the fibrin microthread is approximately 1 CM.

4. The method of claim 1, wherein the v-shaped chamber is comprised of a 2% agarose gel or PDMS.

5. The method of claim 1, wherein the dimensions of the v-shaped chamber are approximately 1.5 cm×0.75 cm×0.5 cm.

6. The method of claim 1, wherein the cells are human mesenchymal stem cells.

7. The method of claim 1, wherein the period of time is four hours.

8. The method of claim 1, wherein the number of cells is 10,000-50,000.

9. A method of generating small-diameter blood vessels, the method comprising:
injecting a collagen gel into a mold aligned coaxially around a silicon tube, resulting in a collagen-coated tube;
securing the collagen-coated tube to a silicon ring adhered to a culture dish;
resuspending a number of cells in a growth medium to create a suspension;
adding an amount of the suspension to the center of the silicon ring;
inverting the culture dish for a first period of time, such that the suspension comes in contact with the collagen-coated tube, resulting in a seeded tube;
turning the culture dish upright;
rinsing the seeded tube at least twice with an amount of saline;
adding a second amount of the growth medium to the seeded tube;
incubating the seeded tube for a second period of time, resulting in a tissue-engineered blood vessel; and
removing the tissue engineered blood vessel from the silicon ring.

10. The method of claim 9, wherein the gel is a rat tail type I collagen gel mixed with 1N NaOH and 5X DMEM saturated with sodium bicarbonate.

11. The method of claim 9 wherein the cells are adult rat aortic smooth muscle cells.

12. The method of claim 9, wherein the growth medium is Dulbecco's Modified Eagle Medium.

13. The method of claim 9, wherein the concentration of the suspension is 1 million cells/ml.

14. The method of claim 9, wherein the amount of suspension is 280 microliters.

15. The method of claim 9, wherein the first period of time is 30 minutes.

16. The method of claim 9 wherein the second amount of growth medium is 6 ml.

17. The method of claim 9, wherein the second period of time is 14 days.

18. A tissue engineered blood vessel as made by the method of claim 1.

19. A tissue engineered blood vessel as made by the method of claim 9.
